(19) United States
(12) Patent Application Publication
(10) Pub. No.: US 2011/0112017 A1
(43) Pub. Date: May 12, 2011

(54) COMPOSITIONS, METHODS AND USES FOR TREATING DIABETES

(76) Inventors: Lee SHAPIRO, Denver, CO (US); Charles A. DINARELLO, Boulder, CO (US); Eli LEWIS, Rehovot (IL); Peter GOTTLIEB, Boulder, CO (US); Amy WALLACE, Boulder, CO (US); Aaron MICHELS, Aurora, CO (US); Gregory B. POTTS, Boulder, CO (US)

(21) Appl. No.: 12/814,263

(22) Filed: Jun. 11, 2010

Related U.S. Application Data
(63) Continuation-in-part of application No. 09/518,081, filed on Mar. 3, 2000, now Pat. No. 7,704,958.

(51) Int. Cl. A61K 38/17; A61P 3/10

(52) U.S. Cl. 514/7.3

(57) ABSTRACT

Embodiments herein concern compositions and methods for treating diabetes in a subject.

### PRIORITY

This application claims priority to U.S. patent application Ser. No. 09/518,081 filed Mar. 3, 2000, which issued as U.S. Pat. No. 7,704,958 on Apr. 27, 2010. In addition, this application claims priority to provisional application U.S. application Ser. No. 60/123,167, filed Mar. 5, 1999. Both applications are incorporated by reference in their entirety.

### FIELD OF USE

Embodiments herein report methods, compositions and uses for treatment of a subject having diabetes. Embodiments of this application also generally report methods, compositions and uses of alpha-1 antitrypsin (AAT) to treat a subject having Type 1 diabetes. In some embodiments, the subject having Type 1 diabetes may have detectible c-peptide levels and/or detectible insulin production and/or residual islet cell function. In certain embodiments, compositions and methods herein concern treating a subject having Type 1 diabetes for a short duration.

### BACKGROUND

There are two types of diabetes, Type 1 and Type 2. Type 1 diabetes (T1D) is the immune mediated form of diabetes. It is an autoimmune disease that is organ specific for the pancreatic beta cells. The disease pathogenesis involves T-cell infiltration into the islets of the pancreas that subsequently destroys the insulin producing cells, and result in overt symptoms of disease. Currently there is no known cure for T1D and treatment for the disease consists of lifelong administration of insulin. Despite treatment with insulin therapy long-term complications, including nephropathy, retinopathy, neuropathy, and cardiovascular disease, can result.

Currently, there are no treatments available for preservation of insulin production outside of research studies. Previously, the combination of cyclosporine and azothioprine plus glucocorticoids was studied and found to have unfavorable side effect profiles. Studies using anti-CD3 monoclonal antibodies have demonstrated the ability to slow the beta cell destruction in recent onset T1D. Again, the anti-CD3 studies also had serious adverse events and induce immunosuppression. Several antigen studies are also under way. Previously, antigen therapy with oral insulin demonstrated a delay in onset of T1D in subjects at increased risk for developing T1D. Several other antigens are also being studied in new onset T1D including proinsulin DNA vaccines and GAD65 vaccines.

### SUMMARY

Embodiments herein report methods, compositions and uses for treatment of diabetes in a subject. In some embodiments, a subject can be a subject having Type 1 diabetes (T1D). In accordance with these embodiments, Type-1 diabetics can be subjects having been diagnosed in a certain time period having remaining insulin production. In these examples, insulin production can be about 1 to about 10 pmols/ml or about 2 to about 5 pmols/ml insulin production by the subject. In other embodiments, a subject may be treated with compositions disclosed herein to modulate destruction of islet cell function. In other embodiments, compositions and methods disclosed herein may be used to treat Type 1 diabetics for about 5 weeks to about 2 years, or about 8 weeks to about 1 year etc. with compositions disclosed herein. Thus, treatment may be for a short duration compared to long term treatment using insulin replacement therapy.

### BRIEF DESCRIPTION OF THE DRAWING

FIG. 1 illustrates the effect of α1-antitrypsin and the peptoid CE-2072 on apoptosis in RCG Neuron (rat cerebral granule) cells, also termed RCGC.

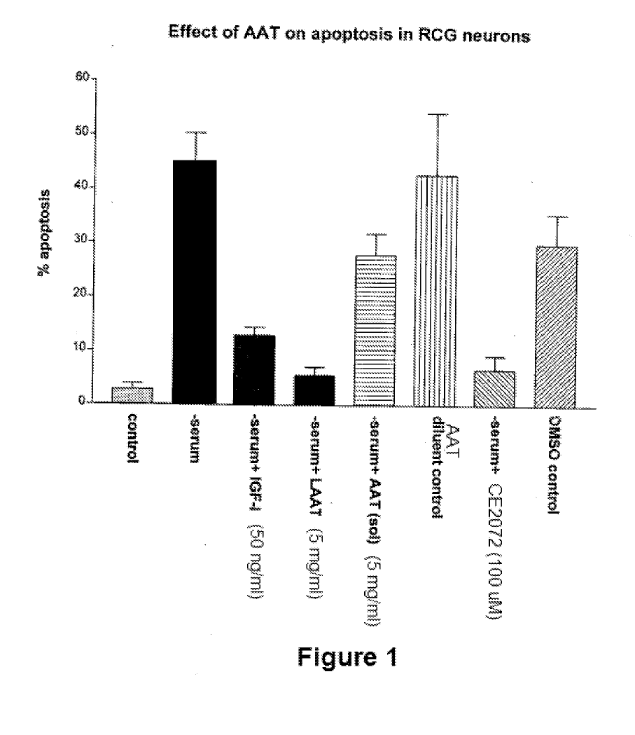

FIG. 2 illustrates short term AAT treatment of diabetic NOD mice.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

Type 1 diabetes is an immune-mediated form of diabetes. It is an autoimmune disease that is organ specific for the pancreatic beta cells. The disease pathogenesis involves T-cell infiltration into the islets of the pancreas that subsequently destroys the insulin producing cells, and result in overt symptoms of disease. While the progress to complete insulin dependence can occur quickly after clinical onset, initially after diagnosis the pancreas is able to produce a significant amount of insulin (the “honeymoon” period). During this state, a subject will have less glucose variability. The Diabetes Control and Complications Trial (DCCT) found that 20% of patients studied, who were within 5 years of diagnosis, had remaining insulin production (2-5 pmol/ml). At this time immunologic intervention can potentially save beta cell function and reduce reliance on insulin. With the increasing incidence of T1D, therapies aimed at altering the underlying autoimmune process need to be investigated. Even partial beta cell function is beneficial as patients that maintain endogenous insulin production have better metabolic control than those who rely solely on exogenous insulin and improved metabolic control reduces the long-term complications from diabetes. Therapies that halt beta cell destruction can lead to continued endogenous insulin production, greatly improving metabolic control and reducing complications in T1D.

In some embodiments, a subject having Type 1 diabetes (T1D) with recent onset T1D having residual beta cell function may be treated with alpha 1 antitrypsin (AAT). AAT is a serine protease inhibitor that has been used safely since 1987 in humans. It is known that AAT deficiency can lead to emphysema and liver cirrhosis in humans. AAT has been shown to have anti-inflammatory effects and potentiate immune tolerance.

In certain embodiments, AAT-associated molecules used in the methods and compositions herein can include, but are not limited to, compositions of naturally occurring AAT (394 AA length molecule making up approximately 90% of AAT isolated from serum). These compositions may be partially or fully purified compositions of AAT molecules.

In other embodiments, α1-antitrypsin used in the methods and compositions herein can be a commercially available composition and can include, but is not limited to, Aralast™ (Baxter), Zemaira™ (Aventis Behring), Prolastin™ (Bayer), AAT (Kamada Ltd. Israel) or any other commercially available composition or any combination thereof. In other embodiments, AAT used in methods and compositions herein can include naturally-occurring or a mutant form of AAT purified or partially purified from a mammalian source.

In some embodiments, pharmaceutical compositions contemplated herein are administered orally, systemically, via an implant, intravenously, topically, intrathecally, intracranially, intraventricularly, by inhalation or nasally. In certain embodiments, inhalable AAT may be manufactured by Kamada (Israel).

In certain embodiments, synthetic and/or naturally occurring peptides/proteins may be used in compositions and methods herein for example, providing other than serine protease inhibitor activity of AAT. Homologues, natural peptides, or peptidyl derivatives, e.g., aldehyde or ketone derivatives of such peptides are also contemplated herein. Without limiting to AAT, compounds like oxadiazole, thiadiazole and triazole peptoids and substances can include, but are not limited to, certain phenylenedialkanoate esters, CE-2072, UT-77, and triazole peptoids. Examples of analogues are TLCK (tosyl-L-lysine chloromethyl ketone) or TPCK (tosyl-L-phenylalanine chloromethyl ketone) or any combination thereof.

In certain embodiments, AAT could potentially inhibit the inflammation associated with T1D and/or enhance immune tolerance resulting in sustained beta cell function. With its long term pharmacologic use along with the above data on inflammation in T1D and the pre-clinical studies in the NOD mouse model, AAT is likely a safe and potentially beneficial therapy for example, for recent onset type 1 diabetics.

### Proteins

In certain embodiments, proteins may be purified or partially purified and used as compositions for methods disclosed herein. Proteins contemplated of use herein can include, but is not limited to, purified or partially purified compositions of AAT, for example, naturally-occurring forms of AAT or synthetically made compositions of AAT.

### Pharmaceutical Compositions:

In one embodiment, the compound (e.g. pharmaceutical chemical, protein, gene, antibody, or anti-viral agent) may be administered to a subject in need thereof subcutaneously, intravenously, by oral administration, inhalation, transdermally, intravaginally, topically, intranasally, rectally or a combination thereof. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be administered intravenously. In one particular embodiment, the compound may be administered intranasally, such as inhalation.

### Kits

In certain embodiments, a kit contemplated herein may include compositions for treating a subject having diabetes. In certain embodiments, kits may include a short-duration supply of AAT for treatment of a subject having Type 1 diabetes.

The instant application incorporates herein by reference in their entirety for all purposes, the related applications claimed in the priority section above (e.g. U.S. Pat. No. 7,704,958 issued Apr. 27, 2010 and U.S. Provisional Application No. 60/123,167, filed Mar. 5, 1999).

### EXAMPLES

Examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

### Example 1

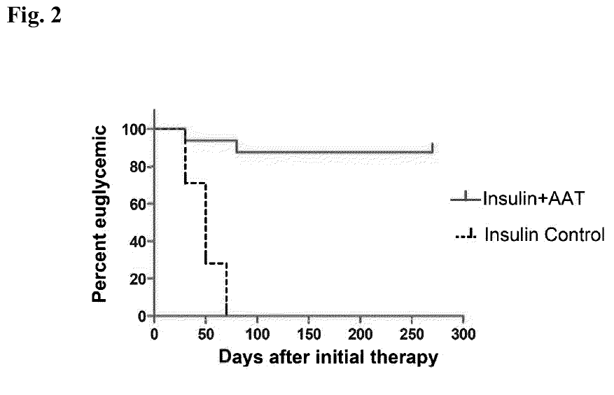

In one study, diabetic NOD mice with blood glucoses between 300 and 450 mg/dl were treated with a short 2 week course of human AAT. 88% (21/24) of mice remained euglycemic for >270 days compared to the controls that were treated with insulin therapy (FIG. 2). FIG. 2 illustrates short term AAT treatment of diabetic NOD mice restores euglycemia. AAT treated mice were compared with insulin control mice by using Wilcoxon signed rank test (P<0.0001).

It was also observed that some subjects had decreased inflammatory mediators in the pancreatic lymph nodes and pancreata of AAT treated mice compared to control NOD mice.

### Example 2

In a proposed clinical study: treat with Alpha₁-antitrypsin (AAT) weekly for 8 weeks in subjects with type 1 diabetes and detectable c-peptide will:
1. Be a safe and feasible treatment for people with diabetes (Type 1 or potentially Type 2)
2. Delay the loss of c-peptide
3. Sustain metabolic control of glucose levels as measured by HbAlc and glucose fluctuations

### Aims:

1. To assess participant safety
2. To assess treatment feasibility

### Aims:

1. To assess the effects of AAT on the maintenance of C-peptide production
2. To assess the effects of AAT on glycemic variability and HbAlc
3. To begin to examine the underlying mechanisms for how AAT may effect T1D progression
4. To assess the safety and tolerability of AAT in subjects with T1D at a dose of 80 mg/kg.

Description of a Population to be Enrolled in a Diabetes Clinical Trial: Study Design and Research Methods

### Certain Criteria:

Cohorts of 10 subjects who are all within 5 years from diagnosis with T1D and continue to produce insulin will be enrolled. The Diabetes Control and Complications Trial (DCCT) found that 20% of patients studied, who were within 5 years of diagnosis, had remaining insulin production (2-5 pmol/ml). This study will enroll participants who have a stimulated c-peptide of ≧0.2 pmol/mL. This is the same inclusion criteria stipulated in other similar studies where the clinical outcome is preserving remaining insulin production.

The investigators will evaluate safety and efficacy between each cohort of 10 subjects. The enrolled population will still have residual beta cell function and will be producing some of their own insulin. This will enable us to test the hypothesis that AAT can slow the loss of c-peptide production. As well, by enrolling subjects who are not within the first 90 days of diagnosis, this study will not compete for enrollment with other ongoing studies.

A. Inclusion Criteria:

1. Diagnosis of Type 1 Diabetes Mellitus based on ADA Criteria for fewer than 5 years but More Than 100 days
2. 6-46 years of age, inclusive. To assess safety, 3 patients over the age of 16 will be enrolled. The first 3 patients will be staggered by 2 weeks, and following the last infusion of the final patient, we will assess adverse events. As long as there are no stopping criteria met for these 3 patients the age criteria will go down to 6 years old.
3. C-peptide increase during screening mixed meal tolerance test with a minimal stimulated value of ≧0.2 pmol/mL
4. Positive for antibodies to insulin (if insulin autoantibody positive only, determination must be within two weeks of insulin initiation), GAD-65, IA-2 or ZnT8
5. Agree to intensive management of diabetes with an HgbAlc goal of <7.0%

B. Exclusion Criteria:

1. Unable or unwilling to comply with the requirements of the study protocol
2. Body Mass Index (BMI)>30 kg/m2
3. Unstable blood sugar control defined as one or more episodes of severe hypoglycemia (defined as hypoglycemia that required the assistance of another person) within the last 30 days
4. Previous immunotherapy for T1D
5. Administration of an experimental agent for T1D at any time or use of an experimental device for T1D within 30 days of screening, unless approved by the study PI
6. History of any organ transplant, including islet cell transplant.
7. Active autoimmune or immune deficiency disorder (e.g. sarcoidosis, rheumatoid arthritis)
8. Serum bilirubin >ULN, except those Subjects whose abnormal values were attributed to any stable, benign condition (such as Gilbert's Syndrome) may be included
9. Individuals with a history of IgA deficiency
10. Individuals with a history of hypersensitivity to AAT

### AAT Treatment:

Eligible subjects will be treated once a week for 8 weeks (8 total treatments). AAT will be provided by Baxter pharmaceuticals. AAT at 80 mg/kg will be infused at a rate of 0.08 ml/kg body weight per minute. A 50 kg subject would receive 4000 mg AAT. A 1000 mg vial of AAT is diluted in 50 mls. The dose of 4000 mg dilutes to 200 mls. At a rate of 0.08 ml/kg/min the subject will receive 4 ml/min. A 200 ml infusion at 4 ml/min would take 50 min. Typically, these infusions will last under 1 hour. Infusions will take place at the outpatient CTRC. All subjects will receive study drug, this is an open label trial.

Participants will not be pretreated with any medication; however acetaminophen and diphenhydramine will be available at the bedside for treatment of any reactions. Vitals will be taken prior to AAT administration and for every 30 minutes for the duration of the infusion. CBC, chemistry and urine pregnancy (if female) tests will be done and reviewed prior to each infusion.

Patients will be contacted daily for 2 days following each infusion to assess adverse events.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a subject comprising,
identifying a subject having Type 1 diabetes;
identifying the subjects having Type 1 diabetes that produce insulin; and
administering a therapeutically effective amount of a composition comprising alpha-1 antitrypsin (AAT) to the subject, wherein the composition modulates beta cell destruction in the subject.

2. The method of claim 1, wherein insulin production in the subject is around 1 pmol/ml to about 10 pmol/ml.

3. The method of claim 1, wherein the subject has residual beta islet cell function.

4. The method of claim 1, further comprising combining the composition with at least one other therapeutic treatment for diabetes.

5. The method of claim 1, wherein the subject is treated with the composition for 2 years or less.

6. The method of claim 1, wherein the subject is treated with the composition for 10 weeks or less.

7. The method of claim 1, wherein treatment of the subject comprises a treatment that is bi-weekly or one time weekly or one time daily.

8. The method of claim 1, wherein the treatment comprises a commercial source of AAT.

9. The method of claim 1, wherein the treatment comprises about 20 mg/kg to about 100 mg/kg of AAT to the subject.

10. A method for treating a subject comprising,
identifying the subjects having Type 1 diabetes;
identifying the subjects having stimulated c-peptide of >0.2 pmol/mL; and
administering a therapeutically effective amount of a composition comprising alpha-1 antitrypsin (AAT) to the subject, wherein the composition modulates beta cell destruction in the subject.

11. A method for treating diabetes in a subject comprising administering to the subject a therapeutically effective amount of a composition comprising AAT or a derivative thereof.

12. The method of claim 11, wherein the composition comprises naturally occurring AAT.

13. The method of claim 11, wherein the AAT is substantially purified from a wild type, mutant, or transgenic mammalian source.

14. The method of claim 11, wherein the AAT is isolated from a culture producing wild type, mutant, or transformed cells.

15. The method of claim 11, wherein the amount of AAT or the derivative thereof in the composition ranges from about 1 mg/kg to about 100 mg/kg of body weight of the subject.

16. The method of claim 11, wherein the composition is administered parenterally, orally, nasally, buccally, intravenously, intramuscularly, subcutaneously, intrathecally, transdermally, by osmotic pump, by inhalation, or a combination thereof.

17. The method of claim 11, wherein the treatment delays the loss of c-peptide in the subject.

18. The method of claim 11, wherein the composition is administered at least once weekly or once daily.

19. The method of claim 11, wherein the subject has Type 1 diabetes and has detectible c-peptide levels.
